(19) United States
(12) Patent Application Publication
(10) Pub. No.: US 2026/0306595 A1
(43) Pub. Date: Oct. 8, 2026

(54) ZONAL AND TARGETED METHODS AND USES FOR TREATING PAIN, INFLAMMATION, STIFFNESS AND ARTHROFIBROSIS AND BALANCING MUSCLE FORCES AFTER TOTAL KNEE ARTHROPLASTY (TKA)

(71) Applicant: ARCK Biologics, Inc., Winston Salem, NC (US)

(72) Inventor: Louis Andrew Koman, Winston-Salem, NC (US)

(21) Appl. No.: 19/641,686

(22) Filed: Apr. 8, 2026

Related U.S. Application Data
(60) Provisional application No. 63/785,260, filed on Apr. 8, 2025.

(51) Int. Cl. A61K 38/48; A61K 9/00

(52) U.S. Cl. CPC A61K 38/4893; A61K 9/0019

(57) ABSTRACT

A method for treating and preventing postoperative pain, inflammation, stiffness, and arthrofibrosis following total knee arthroplasty (TKA), and for balancing muscle forces to maintain range of motion and improve long-term outcomes, comprises the intraoperative administration of a botulinum toxin through a comprehensive, multi-zonal targeting approach. In the preferred embodiment, approximately 400 units of onabotulinumtoxinA or equivalent are reconstituted in approximately 10 cc of sterile preservative-free saline at a concentration of approximately 40 units per cc, and are administered across four zones: approximately 60 units intraarticular (single injection through closed capsule after watertight closure, diffusing throughout the joint space); approximately 60 units intracapsular (administered to synovial tissue under direct visualization, timing flexible); approximately 80 units extracapsular and interstitial (targeting genicular nerve exit and entry points via an advance-and-withdraw needle technique at approximately 1 to 2 cm intervals through 2 to 3 portals); and approximately 200 units intramuscular (rectus femoris and biceps femoris in all cases; semimembranosus and semitendinosus when tight; gastrocnemius optionally when contracture is identified; gracilis rarely). All injections are performed under direct vision or ultrasound guidance at a slow injection rate not exceeding approximately 0.5 cc per 3 to 5 seconds. The method is designed to reduce postoperative pain, preempt the inflammatory and fibrotic cascade before it becomes established, prevent stiffness and arthrofibrosis, maintain range of motion during rehabilitation, reduce the need for manipulation under anesthesia, and improve long-term functional outcomes.

### CROSS-REFERENCE TO RELATED APPLICATIONS

This application claims priority to and the benefit of U.S. Provisional Patent Application Ser. No. 63/785,260, filed Apr. 8, 2025, the entire contents of which are incorporated herein by reference.

### FIELD OF THE INVENTION

This invention relates to orthopedic surgery and clinical pharmacology, and more particularly to methods and uses for the intraoperative administration of botulinum toxin during total knee arthroplasty (TKA) to treat and prevent postoperative pain, inflammation, stiffness, and arthrofibrosis, and to balance muscle forces to maintain range of motion and improve long-term functional outcomes.

### BACKGROUND

Total knee arthroplasty (TKA) is one of the most commonly performed surgical procedures in the world. In the United States alone, approximately 1.47 million TKA procedures were performed in 2025 with a projected increase of more than 130% by 2040, and more than 450% by 2060. These annual procedure volume projections are based, in part, on population aging, increasing prevalence of osteoarthritis and the outcomes of TKA. While TKA reliably reduces joint pain and improves function in the majority of patients, a clinically significant subset of patients experiences suboptimal postoperative outcomes that adversely affect quality of life and impose substantial economic burdens on the healthcare system.

Postoperative pain following TKA remains a major clinical challenge. Despite the availability of periarticular injection protocols, peripheral nerve blocks, epidural analgesia, and systemic analgesic regimens, a substantial proportion of TKA patients continue to experience significant pain during the early recovery period. Persistent pain contributes to patient dissatisfaction, impairs participation in postoperative rehabilitation, delays functional recovery, and increases reliance on opioid analgesics, the latter presenting its own well-documented risks of dependence, adverse effects, morbidity, and mortality.

Beyond pain, stiffness and arthrofibrosis represent two of the most consequential complications following TKA. Stiffness following TKA is caused by one or more of the following: 1) pain and subsequent decreased motion, 2) inappropriate co-contraction of muscle activity (e.g., excessive rectus or quadriceps activation during hamstring-initiated knee flexion and/or excessive biceps femoris or hamstring activation during quadriceps-initiated knee extension); 3) shortening or contracture of the muscle fibers within the quadriceps or hamstrings; and 4) contracture or arthrofibrosis within the joint capsule or collateral ligaments. Pain and inappropriate co-contracture are more amenable to pharmacological treatment; contracture of muscle and joints requires mechanical correction in addition to medical management. Arthrofibrosis is best managed by prevention.

Arthrofibrosis is a pathological fibrotic response that can follow joint trauma or surgery and is characterized by intraarticular and periarticular proliferation of fibrous tissue, capsular contracture, and loss of range of motion. Studies have demonstrated that approximately 20% of TKA patients experience clinically significant stiffness during recovery. Of these, approximately 4% to 5% require manipulation under anesthesia (MUA), a procedure that itself carries risks including fracture, hemarthrosis, and neurovascular injury, and that is associated with substantial additional cost, patient inconvenience, and psychological burden. Increased symptoms of stiffness create worse functional outcomes and lower patient satisfaction rates.

Flexion contracture, defined as the inability to achieve full passive knee extension, is another well-recognized complication that negatively affects TKA outcomes. The management of post-TKA flexion contracture and arthrofibrosis has historically been limited to physical therapy, serial casting, manipulation under anesthesia, and arthroscopic lysis of adhesions. Published case reports and small case series have described the use of botulinum toxin type A injected postoperatively into the hamstring muscles as a treatment for established flexion contracture following TKA. However, these prior approaches are limited to postoperative management of established contracture in specific muscle groups, and do not address the comprehensive prophylactic and therapeutic potential of intraoperative, multi-zonal botulinum toxin administration.

Current approaches to perioperative pain management and prevention of stiffness in TKA are suboptimal in several critical respects. First, postoperative dosing regimens are empirically derived and not specifically tailored to the periarticular neural anatomy of the knee. Second, preemptive management of stiffness and arthrofibrosis is not addressed by current pharmacological protocols. Both dose and volume of any administered agents are inadequately defined for the complex multi-tissue environment of the postoperative knee. Third, there are no FDA-approved indications for botulinum toxin in TKA, and existing published uses have been primarily reactive rather than prophylactic and have targeted only a limited subset of anatomical structures contributing to postoperative complications. Fourth, current approaches do not adequately address the extracapsular interstitial neural networks, the sensory nerve exit and entry points surrounding the knee, or the multiple muscle groups whose imbalance following surgery contributes to long-term functional impairment. Fifth, existing protocols fail to address the diverse serotypes and commercial preparations of botulinum toxin available and the varying unit calculations required for each, creating practical barriers to implementation.

There exists, therefore, a significant unmet clinical need for a comprehensive, intraoperative, prophylactic protocol utilizing botulinum toxin administered via a targeted, multi-zonal approach to address the full spectrum of postoperative pain, inflammation, stiffness, and arthrofibrosis following TKA, while simultaneously balancing the muscle forces acting across the reconstructed joint to optimize range of motion and long-term functional outcomes.

### SUMMARY OF THE INVENTION

In accordance with certain aspects of an embodiment of the invention, provided herein are methods and uses for treating and preventing perioperative and postoperative pain, inflammation, stiffness, and arthrofibrosis following total knee arthroplasty (TKA), and for balancing muscle forces across the reconstructed knee to maintain and improve postoperative range of motion, reduce the incidence of manipulation under anesthesia, decrease excessive scar formation, and improve long-term functional outcomes.

In one aspect, the invention provides a method for treating and diminishing postoperative symptoms and complications following TKA comprising administering a botulinum toxin intraoperatively during the TKA surgical procedure to one or more of: intraarticular regions, intracapsular regions, extracapsular regions, interstitial regions, periosteal regions, intramuscular regions, and sensory nerve entrance and exit areas, and combinations thereof, within and surrounding the peri-genicular region of the knee.

In another aspect, the invention provides a method comprising a comprehensive, multi-zonal approach to botulinum toxin administration during TKA wherein the method targets at least four anatomical zones: (1) the intraarticular zone; (2) the intracapsular zone including pericapsular and synovial areas; (3) the extracapsular and interstitial zone targeting sensory nerve entrance and exit points; and (4) the intramuscular zone targeting specific muscles whose force imbalance contributes to postoperative stiffness and arthrofibrosis.

In certain embodiments, the total dose of botulinum toxin administered according to the methods disclosed herein using onabotulinumtoxinA or an equivalent is in the range of about 200 units to about 500 units, with a preferred embodiment utilizing about 400 units dissolved in about 10 cc of sterile preservative-free injectable 0.9% sodium chloride solution at a concentration of about 40 units per cc.

In certain embodiments, the intraoperative administration of botulinum toxin according to the methods disclosed herein is expected to: (a) decrease postoperative pain; (b) prevent or reduce stiffness during rehabilitation; (c) reduce arthrofibrosis formation; (d) maintain range of motion during post-operative rehabilitation; (e) significantly decrease the need for manipulation under anesthesia; (f) decrease excessive scar formation; (g) balance muscle forces acting across the knee; and (h) improve long-term functional outcomes.

In certain embodiments, the methods disclosed herein include administration to pericapsular and synovial areas including the suprapatellar, infrapatellar, medial, lateral, and posterior capsule and synovial regions, and periosteum.

In certain embodiments, the methods disclosed herein include administration to muscles for the purpose of balancing muscle forces across the reconstructed knee, including the rectus femoris and biceps femoris muscles, and optionally the semimembranosus, semitendinosus, and gracilis or the vastus intermedius, vastus lateralis, and vastus medialis muscles, and further optionally the medial and/or lateral gastrocnemius when contracture of one or both gastrocnemius heads is identified.

In certain embodiments, the methods disclosed herein include targeting of the neural sensory network of the knee, including the pericapsular genicular afferent and efferent nerves, capsular and pericapsular nerves with afferent and efferent branches to and from the knee, the common and infrapatellar branches of the saphenous nerve, the superior lateral genicular nerve, the superior medial genicular nerve, the inferior lateral genicular nerve, the inferior medial genicular nerve, and the common and recurrent fibular nerve branches. These injections are placed at the terminal nerve endings in order to cleave SNARE proteins, thereby blocking the release of pain mediators including, but not limited to, substance P, glutamate, and calcitonin gene-related peptide (CGRP).

In certain embodiments, the methods disclosed herein utilize type A botulinum toxin serotype preparations, including onabotulinumtoxinA (BOTOX®), incobotulinumtoxinA (XEOMIN®), daxibotulinumtoxinA (DAXXIFY®), or abobotulinumtoxinA (DYSPORT®, administered at approximately 2.5 to 3 times the unit dose of onabotulinumtoxinA), or rimabotulinumtoxinB (MYOBLOC®, administered at approximately 50 times the unit dose of onabotulinumtoxinA, with dilution to achieve adequate injection volume as described herein). Other commercially available botulinum toxin preparations, as well as preparations that may become commercially available in the future, may likewise be suitable for use in the methods disclosed herein, provided that appropriate dose conversion and dilution adjustments are made relative to onabotulinumtoxinA units, as would be understood by one of ordinary skill in the art.

In certain embodiments, all injections are performed under direct visualization except for specifically designated procedures, and injection is performed slowly at a rate of no more than about 0.5 cc per 3 to 5 seconds to optimize toxin contact with target organelles and prevent excessive diffusion. Alternatively, injections may be performed with an inflated tourniquet.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIGS. 1A and 1B are anatomical illustrations of the knee from anterior and posterior views, respectively, depicting the primary genicular nerve branches and sensory nerve exit and entry points targeted by the methods of the present invention.

FIGS. 2A-2D provide a four-panel sequential surgical field illustration depicting the intraoperative injection technique as applied during a standard total knee arthroplasty, from initial surgical approach through intraarticular injection following capsular closure.

FIGS. 3A-3C provide a series of three detailed diagrams of the knee from anterior suprapatellar, anterior infrapatellar, and posterior views, respectively, depicting the extracapsular and interstitial injection zone of the preferred embodiment of the present invention, including needle entry portals, injection pathways, and relevant anatomical avoidance structures.

FIGS. 4A-4E are schematic diagrams illustrating the neuromuscular junction distribution patterns of the intramuscular target muscles, depicting the rectus femoris muscle (FIG. 4A), the biceps femoris muscle (FIG. 4B), the semitendinosus muscle (FIG. 4C), the gracilis muscle (FIG. 4D), and the semimembranous muscle (FIG. 4E).

FIG. 5 is a schematic diagram of the gastrocnemius muscle illustrating the distribution of neuromuscular junctions within the medial and lateral heads and the corresponding injection sites.

FIGS. 6A-6B provide a table summarizing the preferred botulinum toxin dosing protocol by injection zone and by commercial preparation.

FIGS. 7A-7C provide a flowchart depicting the step-by-step injection sequence of the preferred embodiment as integrated into the standard total knee arthroplasty surgical workflow.

### DETAILED DESCRIPTION OF THE PREFERRED EMBODIMENTS

The invention may be understood by referring to the following description and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Descriptions of well-known functions and structures are omitted to enhance clarity and conciseness. The terminology used herein is for the purpose of describing particular embodiments only and is not intended to be limiting of the present disclosure. As used herein, the singular forms “a,” “an,” and “the” are intended to include the plural forms as well, unless the context clearly indicates otherwise. Furthermore, the use of the terms “a,” “an,” etc. does not denote a limitation of quantity, but rather denotes the presence of at least one of the referenced item. The use of the terms “first,” “second,” and the like does not imply any particular order, but they are included to identify individual elements. Moreover, the use of the terms “first,” “second,” etc. does not denote any order of importance, but rather the terms “first,” “second,” etc. are used to distinguish one element from another. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. Although some features may be described with respect to individual exemplary embodiments, aspects need not be limited thereto such that features from one or more exemplary embodiments may be combinable with other features from one or more exemplary embodiments.

### Definitions

As used herein, “total knee arthroplasty” or “TKA” refers to a surgical procedure in which the diseased or damaged articular surfaces of the knee joint, including the distal femur, proximal tibia, and in most cases the inferior patella, are resected and replaced with prosthetic implants. The term as used herein includes primary TKA for any indication including primary osteoarthritis, post-traumatic arthritis, and inflammatory arthritis, as well as revision TKA.

As used herein, “arthrofibrosis” refers to a pathological fibrotic response following joint trauma or surgery characterized by intraarticular and/or periarticular proliferation of fibrous connective tissue, resulting in capsular contracture and limitation of joint range of motion.

As used herein, “manipulation under anesthesia” or “MUA” refers to a procedure in which a patient under general or regional anesthesia has the affected joint forcibly manipulated to break down scar tissue and adhesions in an effort to restore range of motion.

As used herein, “peri-genicular region” refers to the anatomical region of the knee joint and its immediately surrounding soft tissues, including the suprapatellar, infrapatellar, medial, lateral, and posterior compartments of the knee capsule and their associated nerves, muscles, tendons, ligaments, and subcutaneous tissues.

As used herein, “intraarticular” refers to administration within the joint space of the knee, i.e., within the synovial cavity bounded by the joint capsule.

As used herein, “intracapsular” refers to administration within or directly into the joint capsule and its immediately adjacent synovial tissues.

As used herein, “extracapsular” refers to administration outside of the joint capsule but within the immediate periarticular soft tissues, including tissues surrounding the joint capsule and periosteum.

As used herein, “interstitial” refers to administration into the interstitial spaces of the soft tissues surrounding the knee, including subcutaneous and deeper soft tissue planes, targeting sensory nerve exit and entry points.

As used herein, “botulinum toxin” refers to a neurotoxin produced by the anaerobic bacterium Clostridium botulinum, or made recombinantly. The term encompasses the complete toxin complex in its various molecular weight forms (approximately 300 kDa, 600 kDa, and 900 kDa complexes), as well as the isolated neurotoxic component (approximately 150 kDa) unassociated with complex proteins. Modified and re-engineered forms of the toxin are encompassed, including serotypes A, B, C1, D, E, F, and G, as well as analogs, derivatives, homologs, parts, sub-parts, and variants thereof. The term “botulinum toxin” as used herein expressly excludes non-neurotoxins, such as cytotoxic Botulinum toxin serotype C2 and Botulinum toxin serotype C3.

As used herein, the term “unit” or “units” refers to the potency or activity ascribed to a botulinum toxin in internationally standardized mouse units (MU), based on U.S. FDA-approved tests such as the mouse intraperitoneal LD50 test. Unless otherwise specified, units disclosed herein refer to units ascribed to onabotulinumtoxinA (BOTOX®). OnabotulinumtoxinA has a potency that is: (1) equivalent to incobotulinumtoxinA; (2) equivalent to daxibotulinumtoxinA; (3) equivalent to prabotulinumtoxinA; (4) approximately 2.5 to 3 times greater than abobotulinumtoxinA; and (5) approximately 50 times greater than rimabotulinumtoxinB (MYOBLOC®). One pikamole (approximately 10-12 moles) of active botulinum toxin molecules is present per 100 units of onabotulinumtoxinA.

Botulinum toxin is a potent biological neurotoxin produced by Clostridium botulinum existing in seven immunologically distinct serotypes, designated A, B, C1, D, E, F, and G. Each serotype shares a common molecular architecture consisting of a dichain protein complex: a light chain of approximately 50 kDa responsible for the toxic enzymatic activity of the molecule, and a larger polypeptide chain of about 100 kDa which is believed to be necessary to enable the toxin to penetrate the SNARE-containing protein structure of a nerve. The two chains are linked by disulfide bridges. The neurotoxic component has a molecular weight of approximately 150 kDa; in native form the toxin exists in non-covalent complexes with associated non-toxic proteins at molecular weights of approximately 300, 600, and 900 kDa depending on serotype and preparation conditions.

Botulinum toxin acts by binding rapidly and with high affinity to presynaptic structures that contain neurotransmitters within vesicles that require SNARE proteins (SNAP-25, syntaxin and synaptobrevin) to fuse to the presynaptic membrane, release the neurotransmitter into the pre-synaptic cleft and attach to the appropriate postsynaptic receptor initiating a reaction. For example, at the sensory terminal nerve ending, the release of pain modulators such as substance P, glutamate and CRGP occurs; however, if a botulinum toxin cleaves SNARE proteins, the pain modulators are unable to be released. Within muscle, cleaving SNARE proteins at the neuromuscular junction prevents the release of acetylcholine and produces flaccid paralysis and within microvascular beds, preventing the release of alpha adrenergic neurotransmitters modifies microvascular perfusion. The pharmacological mechanism of this inhibition involves the enzymatic cleavage of soluble N-ethylmaleimide-sensitive factor attachment receptor (SNARE) proteins required for the docking and fusion of acetylcholine-containing vesicles with the presynaptic terminal membrane. Specifically, botulinum toxin serotypes A, C, and E cleave synaptosomal-associated protein 25 (SNAP-25), while serotype C also cleaves syntaxin, and serotypes B, D, F, and G cleave vesicle-associated membrane protein (VAMP, also known as synaptobrevin). The active molecule is transported into the intracellular domain of the presynaptic terminal where it irreversibly and enzymatically inactivates these SNARE proteins, preventing vesicle fusion and neurotransmitter release. The result is a local, reversible inhibition of neurochemical transmission that does not result in degeneration of neural or muscular tissue and that persists until new SNARE proteins are synthesized and new nerve terminal sprouting occurs, typically over a period of approximately three to six months in most clinical applications.

While the muscle-paralyzing properties of botulinum toxin have been most extensively studied, it is increasingly recognized that the therapeutic effects of botulinum toxin extend beyond motor function. Botulinum toxin targets multiple SNARE-containing organelles in addition to neuromuscular junctions, including nociceptors, sweat glands, vascular beds, and other autonomic structures. Administration of botulinum toxin to sensory nerves inhibits the peripheral release of pain-associated neuropeptides, including substance P, glutamate, and calcitonin gene-related peptide (CGRP), contributing to analgesic effects that are independent of and extend beyond any motor paralysis produced. These sensory inhibitory properties are of particular relevance to the use of botulinum toxin in the management of postoperative pain and neurogenic inflammation following TKA. Additionally, emerging evidence supports an anti-inflammatory role for botulinum toxin in joint and periarticular tissue. Botulinum toxin type A has been shown in randomized controlled trials to treat knee osteoarthritis without negatively impacting cartilage or soft tissue, and has demonstrated anti-inflammatory effects in murine models of arthritic joint disease. Comparative meta-analysis has further demonstrated the efficacy of intraarticular botulinum toxin type A relative to corticosteroid and hyaluronic acid for painful knee osteoarthritis. Modulation of the neurogenic inflammatory cascade, and the attendant reduction in synoviocyte and fibroblast stimulation, is believed to contribute to the reduction in arthrofibrosis formation observed in the clinical application of the methods of the present invention.

The methods of the present invention are distinguished from prior art uses of botulinum toxin in orthopedic surgery by the comprehensive, multi-zonal targeting approach employed, the intraoperative timing of administration, and the simultaneous treatment of multiple distinct anatomical compartments contributing to postoperative pain, inflammation, stiffness, and arthrofibrosis. Prior published uses of botulinum toxin in TKA have been limited to postoperative, reactive management of established flexion contracture through injection into specific hamstring muscles. By contrast, the present invention addresses the full periarticular anatomical landscape of the knee through simultaneous intraoperative targeting of four distinct zones: (1) the intraarticular compartment; (2) the capsular and pericapsular zone; (3) the extracapsular and interstitial zone; and (4) the intramuscular zone. This multi-zonal approach is designed to address concurrently the multiple pathophysiological pathways contributing to postoperative TKA complications: the inflammatory cascade within the joint space, targeted by intraarticular administration; the capsular fibrotic response and nociceptive signaling from capsular nerve endings, targeted by intracapsular and pericapsular administration; the sensory nerve networks that mediate pain transmission and neurogenic inflammation in the periarticular soft tissues, targeted by extracapsular and interstitial administration to genicular nerve exit and entry points; and the muscle force imbalance that promotes stiffness and contracture during rehabilitation, targeted by intramuscular administration to specific muscles of the thigh and, when indicated, the leg. The intraoperative timing of administration is critical to the therapeutic effect of the protocol on all of these pathways. Performing the injections during the surgical procedure allows direct visualization of injection sites and enables precise targeting of anatomical locations that are otherwise inaccessible or difficult to localize percutaneously. More fundamentally, intraoperative administration establishes a pharmacological environment at the moment of tissue injury and surgical trauma, preempting the initiation of fibrotic and inflammatory cascades before they become established. Decreasing immediate postoperative pain and inflammation enhances non-narcotic multimodal interventions and decreases the potential negative effects of pain, narcotic use, and potential addiction. In addition, intraoperative administration blocks, in part, the inflammatory cascade that leads to arthrofibrosis, chronic pain, impaired function, stiffness, joint contracture, instability, and the need for additional surgery. This preemptive approach fundamentally distinguishes the methods of the present invention from prior postoperative, reactive uses of botulinum toxin in TKA.

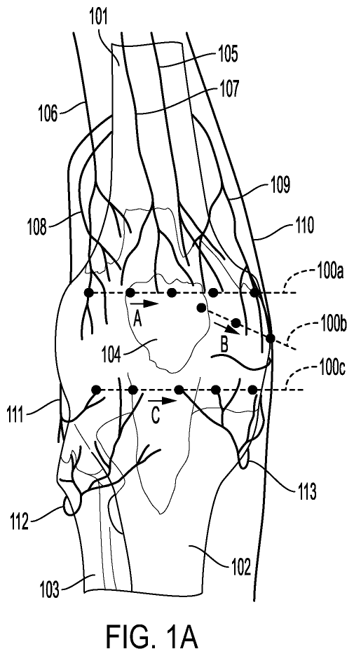

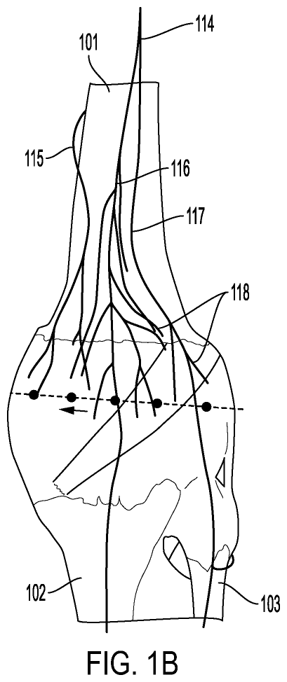

With reference to FIGS. 1A and 1B, the knee joint receives its sensory innervation from a network of genicular nerve branches arising from the femoral, obturator 115, tibial 116, and common fibular nerves 117, as depicted in FIG. 1B. The superior lateral genicular nerve 108 arises from the common fibular division of the sciatic nerve 114 or directly from the common fibular nerve 117, courses along the lateral aspect of the femur 101 superior to the lateral femoral condyle, and provides sensory innervation to the superolateral capsule, the lateral retinaculum, and the superolateral joint structures. The superior medial genicular nerve 109 arises from the tibial division of the sciatic nerve 114 or from the tibial nerve 116, courses along the medial aspect of the femur 101 superior to the medial femoral condyle, and provides sensory innervation to the superomedial capsule, the medial retinaculum, and the superomedial joint structures. The inferior lateral genicular nerve 111 arises from the common fibular nerve 117 or its branches, courses inferiorly along the lateral aspect of the knee below the lateral femoral condyle and above the fibular head 103, and provides sensory innervation to the inferolateral capsule, the lateral collateral ligament region, and the inferolateral joint structures. The inferior medial genicular nerve 113 arises from the tibial nerve 116, courses inferiorly along the medial aspect of the knee below the medial femoral condyle, and provides sensory innervation to the inferomedial capsule and inferomedial joint structures. The saphenous nerve 110, the largest sensory branch of the femoral nerve, descends within the adductor canal along the medial aspect of the thigh and gives rise to the infrapatellar branch, which penetrates the sartorius muscle or its fascia at a variable point in the medial knee region and crosses the knee to supply sensory innervation to the anterior, anteromedial, and infrapatellar regions of the knee, the patellar ligament, and the skin of the anterior knee. The infrapatellar branch is a key contributor to anterior knee pain and a primary target of the interstitial zone injections described herein. The common fibular nerve 117 descends along the posterior and lateral aspect of the knee, winding around the fibular head 103, and gives rise to the recurrent fibular nerve branch 112, which ascends from the region of the fibular head 103 to supply the anterolateral capsule and anterolateral knee structures. Care must be taken to avoid direct injection into the common fibular nerve 117 during injections in the lateral and anterolateral zones. Additional afferent and efferent pericapsular nerve branches 118 supply the muscles in the vicinity of the knee, including branches to the vastus intermedius, vastus medialis, vastus lateralis, and the medial and lateral heads of the gastrocnemius. The complex, overlapping, and interdigitating nature of this genicular nerve network means that effective treatment of post-TKA pain and neurogenic inflammation requires comprehensive coverage of multiple nerve branches rather than targeting a single nerve or structure, supporting the multi-zonal approach of the present invention.

FIGS. 1A and 1B additionally show injection sites (shown as filled circles in FIGS. 1A and 1B) with respect to the primary genicular nerve branches of the knee that are targeted by the methods of the invention, indicating the locations at which toxin is administered, particularly at or adjacent to terminal nerve endings to block the release of pain mediators as discussed above. Tracks 100a, 100b, 100c, and 100d reflect the tracks of a needle used to perform the injections, each shown with a medial approach (although a lateral approach may likewise be employed) to position the needle adjacent a collection of terminal branches of sensory nerves. Injection of toxin is performed preferably as the needle is withdrawn (in the direction of arrows A, B, and C) at approximately 1 cm intervals or continuously. Such toxin placement allows toxin to diffuse to terminal nerve endings to cleave SNARE proteins to block the release of pain mediators.

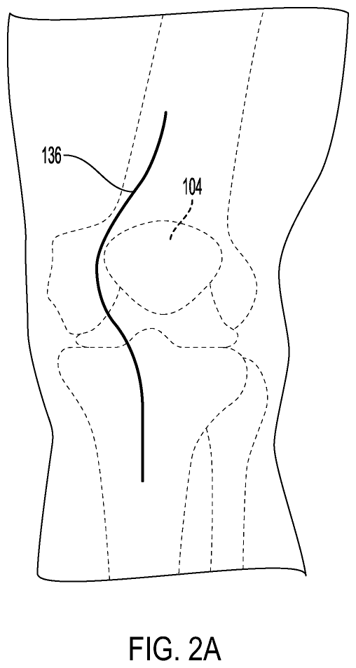

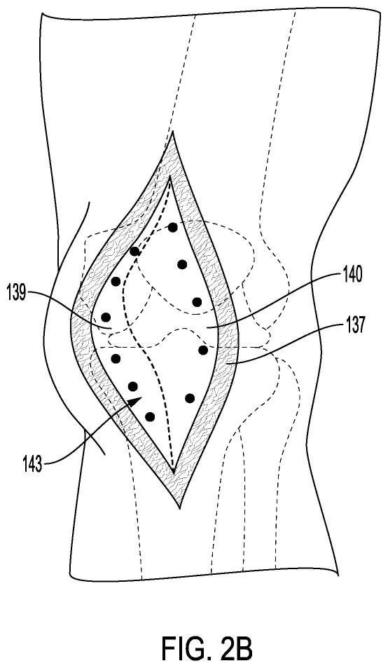

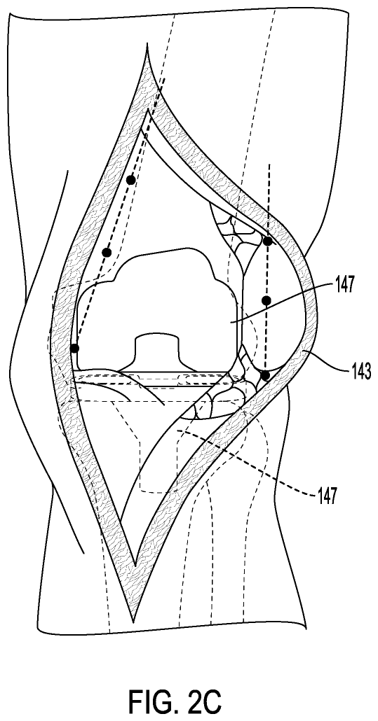

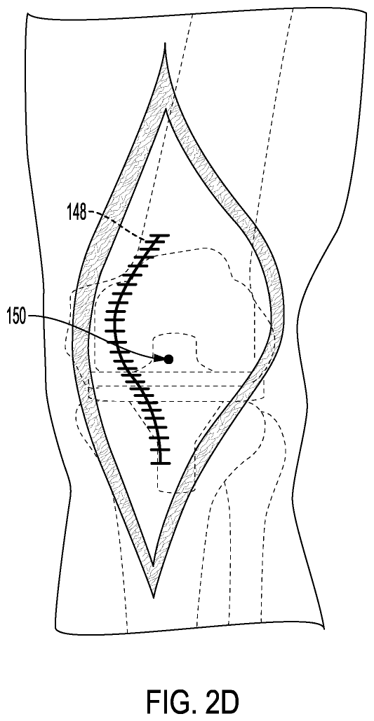

Next and with reference to FIGS. 2A-2D, prior to administration, botulinum toxin is prepared in a sterile field according to the reconstitution protocol described above and drawn into the appropriate syringe or syringes. The standard surgical approach through a median parapatellar incision 136 is depicted in FIG. 2A, with the injection technique as applied at successive stages of the procedure depicted in FIGS. 2B-2D, as described in detail below. In general, no safety cover is placed on the needle during injection, as the safety cap reduces the effective needle length and is not compatible with the injection technique described herein. All injections described herein are performed under direct vision except for specifically designated procedures requiring ultrasound guidance or percutaneous access. The fundamental injection technique for all zones requires slow delivery at a rate not exceeding approximately 0.5 cc per 3 to 5 seconds, which is essential to optimize contact time between the toxin molecules and their target organelles and to prevent excessive diffusion of the toxin to unintended anatomical structures. Aspiration is performed at every injection site prior to injection to confirm non-intravascular needle position. Needle specifications for all zones are as follows unless otherwise stated for a specific zone: 25-gauge, 2-inch standard hypodermic needle, without safety cover. For the rectus femoris intramuscular injection discussed below, a 25-gauge, 4 to 5-inch spinal needle is used to permit distal-to-proximal access along the full length of the accessible muscle belly. The needle is inserted to the appropriate depth for the target zone and injection is performed at depth with multiple discrete injections or as a continuous slow injection preferably during controlled needle withdrawal with the needle advanced to the target depth before injection begins (although injection during needle insertion may optionally be performed where determined necessary due to patient physiology or other factors). All injections are preferably performed at an angle of approximately 45 to 90 degrees relative to the tissue surface, in line with the target muscle, capsule, or tissue plane. In certain embodiments, the complete injection protocol may be performed with a pneumatic tourniquet inflated on the operative extremity, which may assist in localizing the toxin to the peri-genicular region during administration by limiting venous outflow. When a tourniquet is used, the timing of deflation relative to injection completion is at the surgeon's discretion.

The intracapsular injections are administered to the unresected synovial and capsular tissue under direct surgical visualization, as depicted in FIG. 2B and 2C. The timing of intracapsular injection is flexible and may be performed either during the initial surgical approach before implant placement, at the time of capsular closure, or both, at the surgeon's discretion based on intraoperative circumstances. A total of 60 units are administered as injections into the synovial tissue 143 directly at, for example, 1 to 2 cm intervals (with exemplary injection points being depicted as closed circles in FIG. 2B). Alternatively, the continuous injection technique upon needle withdrawal described herein may likewise be employed if access permits, as shown in FIG. 2C (reflecting injections into the medial and lateral synovium). The toxin diffuses throughout the intracapsular space with gentle passive knee motion following administration. As shown in FIGS. 2B-2D, intracapsular injection sites include the medial capsule 139 and synovial area, the lateral capsule 140 and synovial area, the suprapatellar capsule and synovial area, the infrapatellar capsule and synovial area, and the periosteal margins of the resected bony surfaces. The posterior capsule is not directly accessible in most standard surgical approaches; however, the needle may be directed adjacent to the posterior edge of the capsule to the extent permitted by direct visualization. The intracapsular injection technique is not modified for patients with prior surgical procedures.

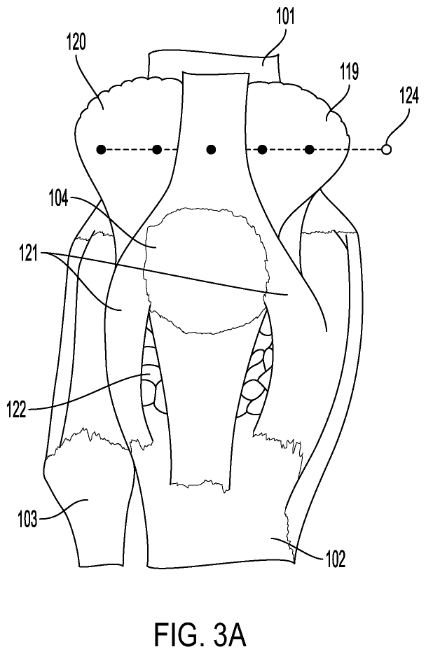

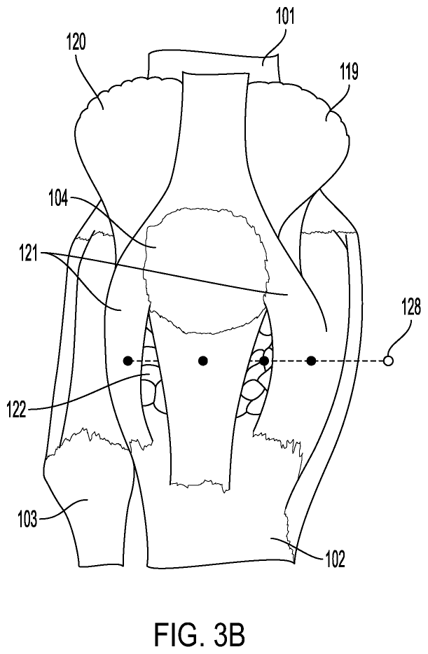

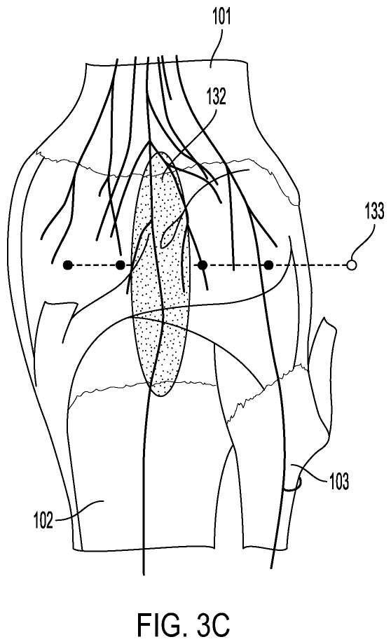

The extracapsular and interstitial injections target the sensory nerve networks outside of the joint capsule that mediate periarticular pain and contribute to the neurogenic inflammatory cascade, as depicted in FIG. 3A-3C. A total of 80 units are delivered to the extracapsular periarticular soft tissues targeting sensory nerve exit and entry points. The extracapsular injection technique preferably employs an advance-and-withdraw approach: the needle is placed at a portal entry point, advanced to the distal extent of the injection zone, aspiration is performed to confirm non-intravascular placement, and injection is administered continuously or at approximately 1 to 2 cm intervals during controlled needle withdrawal. This technique allows comprehensive coverage of the target zone from a limited number of entry portals, typically 2 to 3 portals in total for the full circumferential extracapsular zone. One tissue plane per anatomical area is typically targeted per needle pass. All extracapsular injection sites accessible through the surgical incision 136 are performed under direct vision; sites not accessible through the surgical incision are performed using a spinal needle passed through a separate skin puncture.

The specific extracapsular injection zones are as follows. In the superior lateral genicular nerve 108 zone, the needle is advanced in the tissue plane between the vastus muscles 119, 120 and the superior capsule and fat pad 122, and injection is performed at approximately 1 to 2 cm intervals during withdrawal, targeting the terminal branches of the superior lateral genicular nerve 108. In the superior medial genicular nerve 109 zone, the needle is advanced in the same tissue plane on the medial side, targeting the terminal branches of the superior medial genicular nerve 109. The anterior suprapatellar extracapsular and infrapatellar extracapsular injection zones described above are depicted in FIGS. 3A and 3B, respectively, showing the primary medial entry portals 124, 128, although lateral entry portals may likewise be employed. In the inferior lateral genicular nerve 111 zone, the fibular head 103 and common fibular nerve 117 are identified; the needle is advanced above the capsule 123 and into the fat pad 122 in the region of the inferolateral capsule, with care taken to avoid direct injection into the common fibular nerve 117. Injection is directed into the capsule and fat pad superior to the fibular head 103 rather than into the nerve itself. In the inferior medial genicular nerve 113 zone, the needle is advanced above the medial capsule at the terminal branches of the inferomedial genicular nerve 113, with injection performed into the fat pad and above the capsule. In the infrapatellar branch of the saphenous nerve 110 zone, the needle is directed to the terminal branches of the infrapatellar branch 110 at the medial aspect of the knee just above the capsule. In the common and recurrent fibular nerve branch 112 zone, the fibular head 103 and peroneal nerve 117 are identified and avoided, and the needle is advanced above the capsule and fat pad 122 in the anterolateral region targeting the recurrent fibular nerve branches 112. The posterior capsule injection zone, together with the popliteal vascular structures 132 to be avoided during posterior injection, is depicted in FIG. 3C, showing the lateral entry portal 133 (although again a medial entry portal may likewise be employed). The slow injection rate and aspiration requirements described above apply with particular importance in the interstitial zone because of the absence of fascial or capsular constraints that would otherwise limit diffusion.

Following the extracapsular and interstitial injections, and with reference to Panel D of FIG. 2D, the intraarticular injection is performed after watertight capsular closure is confirmed. A 25-gauge needle is inserted through the closed capsule 148 under direct visualization at the midpoint of the capsular closure in the notch area 150, a location that minimizes the risk of contact with the prosthetic implant components 146, 147. Sixty units of onabotulinumtoxinA (or equivalent) are administered as a single injection directly into the joint space following aspiration to confirm non-intravascular placement. The toxin diffuses throughout the joint cavity with subsequent knee range of motion, and no distribution of the intraarticular dose across multiple sites is required.

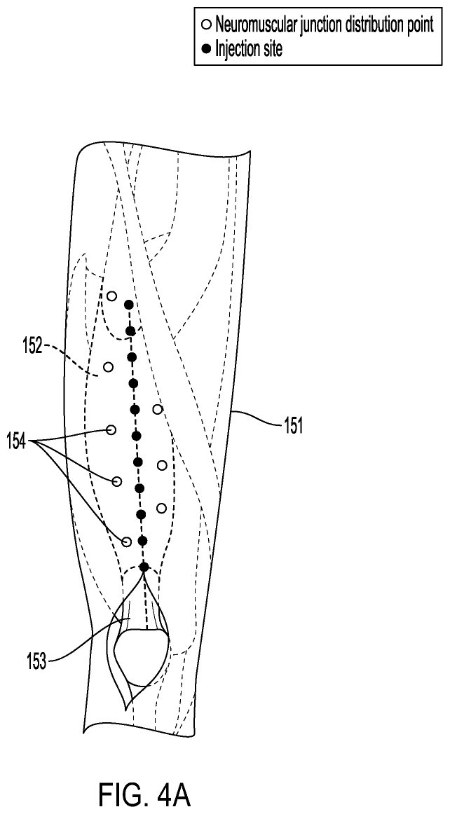

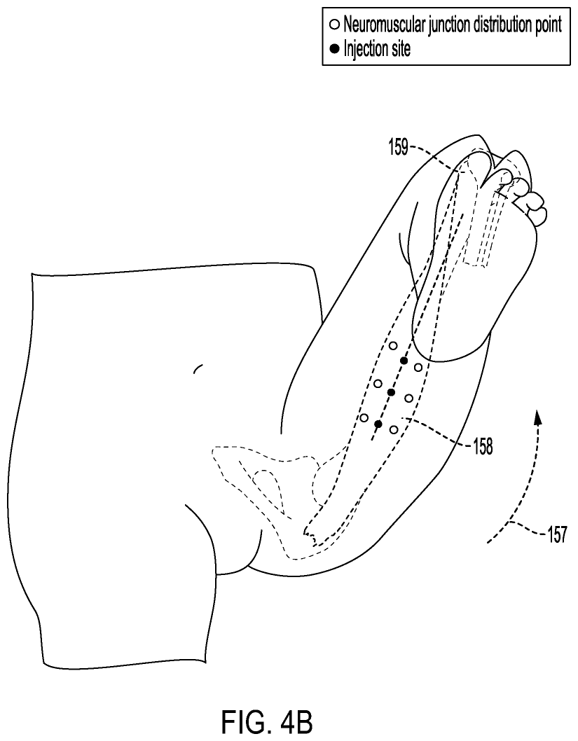

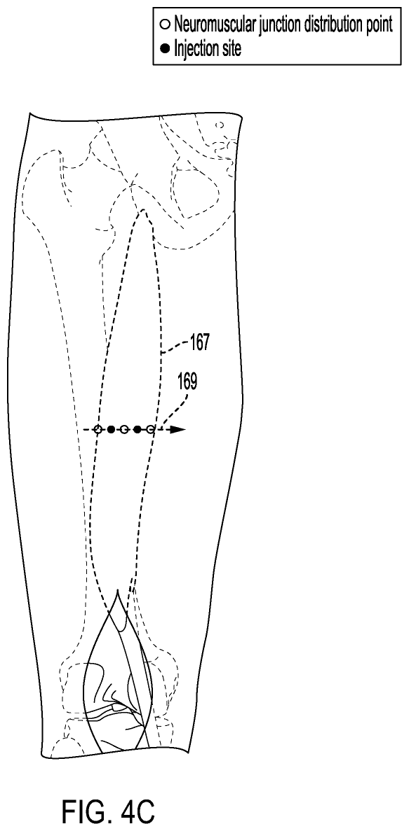

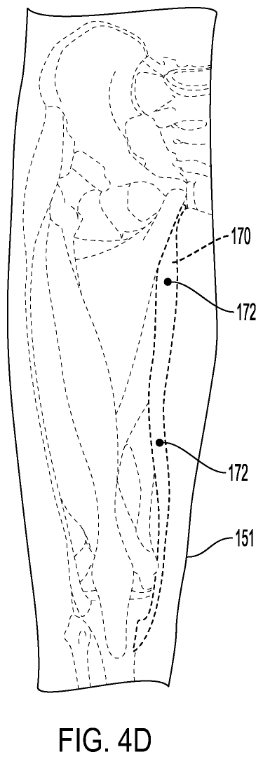

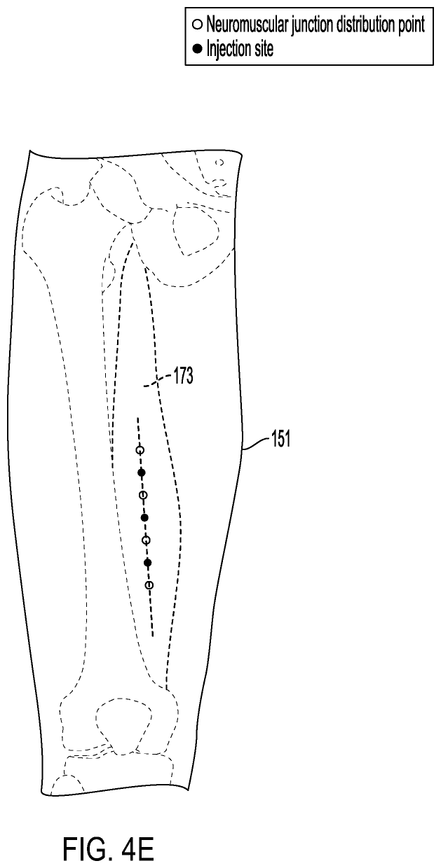

Next and with particular reference to FIGS. 4A-6, the intramuscular injections target specific muscles whose force imbalance across the reconstructed knee joint contributes to postoperative stiffness, flexion contracture, and limitation of range of motion during rehabilitation. The theoretical basis for targeted intramuscular botulinum toxin administration draws on the neuromuscular junction-based dosing principles established in the inventor's prior work (Koman, U.S. Pat. Nos. 8,506,970 and 8,632,786, incorporated herein by reference), which demonstrated that the optimal dose of botulinum toxin for a given muscle is determined by the number and distribution of neuromuscular junctions within that muscle, not by muscle mass or patient body weight. The intramuscular zone receives a total of approximately 200 units distributed among the target muscles as follows.

The rectus femoris 152 (40 units) is the only biarticular muscle of the quadriceps group, crossing both the hip and the knee, and its excessive tension following TKA contributes to difficulty achieving full flexion during rehabilitation. The rectus femoris 152 has two longitudinal bands of neuromuscular junctions 154 running along its length. Injection is performed under direct visualization of the distal portion of the muscle 153 accessible through the standard surgical approach, or alternatively under ultrasound guidance following capsular closure. With reference to FIG. 4A, a 25-gauge, 4 to 5-inch spinal needle is introduced at the distal muscle belly and advanced from distal to proximal into the muscle belly directed along the longitudinal axis of the neuromuscular junction bands. Aspiration is performed and then 40 units are delivered as a slow, continuous injection during controlled needle withdrawal, distributing the full dose along the neuromuscular junction bands 154.

Next and with reference to FIG. 4B, the biceps femoris 158 (40 units) is the lateral hamstring muscle arising from the ischial tuberosity and posterior femur and inserting on the fibular head, and is the primary intraoperative hamstring injection target of the present invention, injected in all standard cases. As depicted in of FIG. 4B, injection is performed following capsular closure with the patient's hip flexed and the knee first flexed and then gently extended to place the muscle 158 under stretch 157. The needle is inserted at the most distal accessible portion of the muscle 158, directed from distal to proximal, and positioned very distal and medial to the common fibular nerve to avoid nerve injury. Aspiration is performed and 40 units are distributed across 2 to 3 injection sites 163 within the NMJ zone. Ultrasound guidance is optional.

Next, and with reference to FIG. 4C, the semitendinosus 167 (60 units) is the second medial hamstring muscle, arising from the ischial tuberosity and inserting on the medial aspect of the proximal tibia 102 via the pes anserinus. It is a unipennate muscle with a single transverse band of neuromuscular junctions 168 at its midbelly, as depicted in FIG. 4C. As with the semimembranosus 173 discussed below, the semitendinosus 167 is injected intraoperatively when tight, or deferred to the early postoperative period under ultrasound guidance when not tight, with 60 units distributed across 2 to 3 injection sites within the injection zone 169 of the transverse neuromuscular junction band 168.

The gracilis 170 (0 to 20 units, optional) is a thin, flat muscle of the medial thigh contributing to the pes anserinus and to medial knee flexion forces. It is a unipennate muscle with two transverse bands of neuromuscular junctions 171. The gracilis 170 injection is rarely indicated in the standard protocol. When gracilis 170 injection is not performed, its 20-unit allocation is added to the biceps femoris 158 dose, bringing the biceps femoris 158 total to 40 units. When gracilis 170 injection is indicated, up to 20 units are administered under ultrasound guidance in the vicinity of the neuromuscular junction zones within the injection zones 172, with a corresponding reduction in the biceps femoris 158 dose, maintaining the 200-unit intramuscular total.

Next and with reference to FIG. 4E, the semimembranosus 173 (60 units) is one of the medial hamstring muscles arising from the ischial tuberosity and inserting on the medial aspect of the proximal tibia, with a relative mass of approximately 0.98 times that of the lateral gastrocnemius 177. The neuromuscular junction distribution of the semimembranosus 173 is longitudinal in orientation, as depicted in FIG. 4E, which should be distinguished from the transverse distribution pattern characteristic of unipennate muscles such as the semitendinosus 167 and gracilis 170. In the preferred embodiment, the semimembranosus 173 is injected intraoperatively when intraoperative assessment reveals the muscle to be tight; otherwise, injection may be deferred to the early postoperative period under ultrasound guidance. When injected, 60 units are distributed across 2 to 3 injection sites along the longitudinal injection pathway with palpation under stretch conditions.

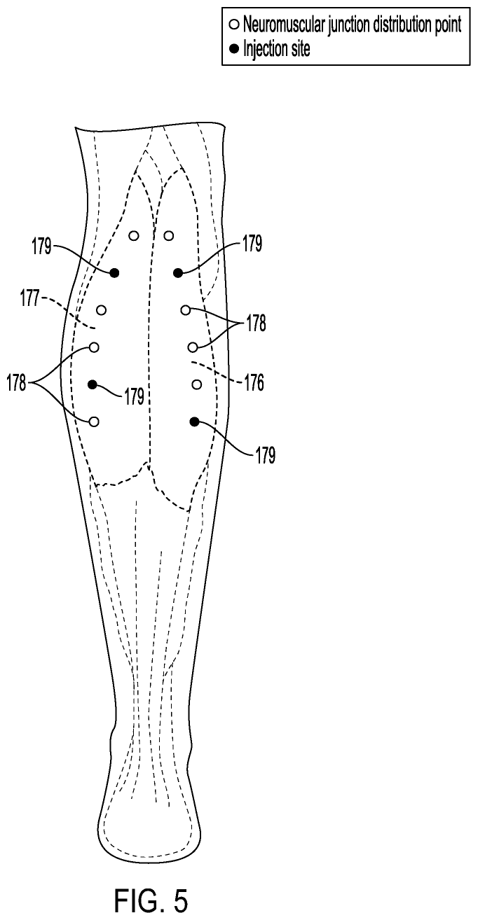

When contracture of the medial 176 and/or lateral 177 gastrocnemius is identified intraoperatively, botulinum toxin may be administered to the affected head or heads. The gastrocnemius muscles have neuromuscular junctions distributed in an inverted U-shaped pattern, as depicted in FIG. 5. When performed, 40 units are administered to the medial gastrocnemius head 176 and 20 units to the lateral gastrocnemius head 177, targeting the neuromuscular junction zone 178 within the inverted U distribution at the injection sites 179 along the injection tracks 180. These doses are reallocated from within the intramuscular zone at the surgeon's clinical discretion based on intraoperative muscle balance assessment, maintaining the 200-unit intramuscular total; in most cases this reallocation is accomplished by deferring the semimembranosus 173 and/or semitendinosus 167 injections to the postoperative period under ultrasound guidance.

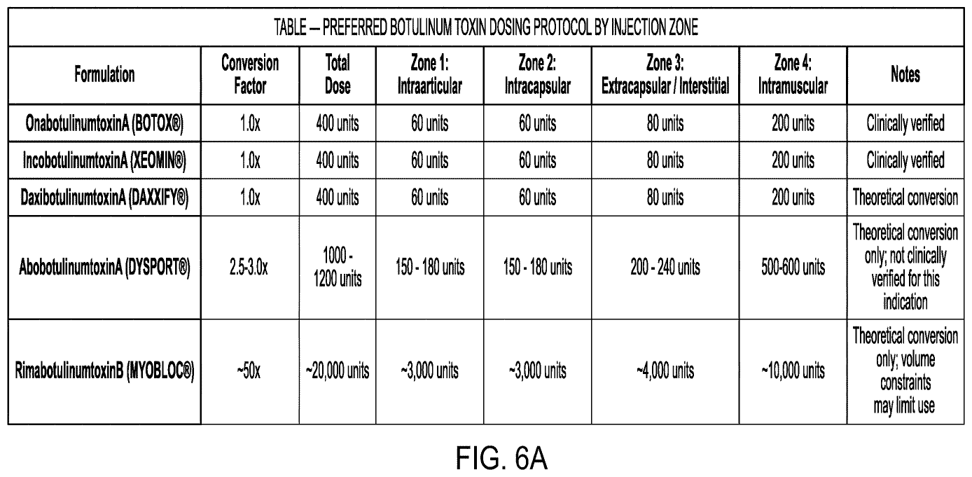

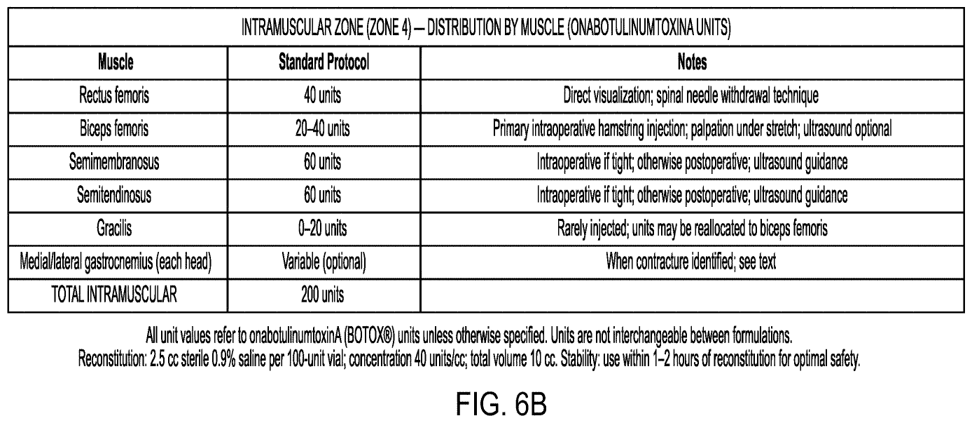

The botulinum toxin used in the methods of the present invention includes any neurotoxin of the genus described above that produces a reversible inhibition of neuromuscular or sensory nerve transmission without degeneration of nervous or muscular tissue, and that is in a pharmaceutically acceptable, sterile form suitable for injection. Commercially available botulinum toxin preparations suitable for use in the methods of the present invention include, without limitation, the following. OnabotulinumtoxinA (BOTOX®, Allergan/AbbVie) is available in sterile, vacuum-dried powder form; each 100-unit vial contains 100 units of Clostridium botulinum type A neurotoxin complex, 0.5 mg of human albumin, and 0.9 mg of sodium chloride, and requires reconstitution with sterile preservative-free injectable 0.9% sodium chloride solution prior to use. OnabotulinumtoxinA has been clinically utilized by the inventor for the TKA injection protocol described herein. IncobotulinumtoxinA (XEOMIN®, Merz Pharmaceuticals) is a purified 150 kDa neurotoxic component of botulinum toxin type A with units equivalent to onabotulinumtoxinA, and has likewise been clinically utilized by the inventor for this protocol. DaxibotulinumtoxinA (DAXXIFY®, Revance Therapeutics) is a botulinum toxin type A preparation with units equivalent to onabotulinumtoxinA, formulated per current manufacturer prescribing information. AbobotulinumtoxinA (DYSPORT®, Ipsen) has a unit potency that is not equivalent to that of onabotulinumtoxinA and requires approximately 2.5 to 3 times the number of units of onabotulinumtoxinA to achieve an equivalent biological effect; units are not interchangeable between DYSPORT® and BOTOX® based on package insert guidelines and suggested dilution tables, and equivalent dosing for this preparation in the methods of the present invention is theoretical. RimabotulinumtoxinB (MYOBLOC®, US WorldMeds) has a unit potency significantly lower than that of onabotulinumtoxinA, requiring approximately 50 times the number of units of onabotulinumtoxinA to achieve an equivalent biological effect; because the commercial preparation of MYOBLOC® may not provide sufficient volume for the multi-zonal injection protocol described herein, dilution with sterile saline may be required prior to use to achieve adequate injection volume, and equivalent dosing for this preparation is theoretical. Other commercially available or future botulinum toxin preparations, including prabotulinumtoxinA (JEUVEAU®), CS-BOT, and PROSIGNE®, may also be used in the methods of the present invention with appropriate unit conversion and dose adjustment. Equivalent dosing for all commercially available preparations discussed herein is provided in the dosing table of FIG. 6. In the preferred embodiments described herein, units refer to onabotulinumtoxinA units unless specifically stated otherwise.

For the preferred embodiment utilizing onabotulinumtoxinA, incobotulinumtoxinA, or daxibotulinumtoxinA, the standard reconstitution protocol is as follows: the contents of each 100-unit vial are dissolved in 2.5 cc of sterile preservative-free injectable 0.9% sodium chloride solution, yielding a concentration of 40 units per cc. For the preferred 400-unit total dose, four 100-unit vials are each reconstituted with 2.5 cc of sterile preservative-free saline, yielding a total volume of 10 cc at a concentration of 40 units per cc. No other agents, including local anesthetics, corticosteroids, or hyaluronidase, are mixed with the botulinum toxin in the same syringe or administered at the same injection site. Following reconstitution, the toxin should be used within 1 to 2 hours for optimal stability and safety; the reconstituted product may be used for up to several hours if necessary, but the 1 to 2-hour window is preferred. The reconstituted toxin is drawn into a sterile 10 cc control syringe in the preferred embodiment; alternatively, multiple 1 cc tuberculin syringes may be used, each pre-loaded with the specific dose for a given zone or muscle to facilitate sequential injection without interruption of the surgical workflow. Alternative reconstitution concentrations may be employed depending on the specific injection zone and the clinical judgment of the administering surgeon; for example, a higher concentration may be preferred for intramuscular injections to limit the volume injected into each muscle, while a lower concentration may be preferred for extracapsular and interstitial injections where broader distribution is desired. The total dose of botulinum toxin remains as specified; only the concentration and injection volume are adjusted.

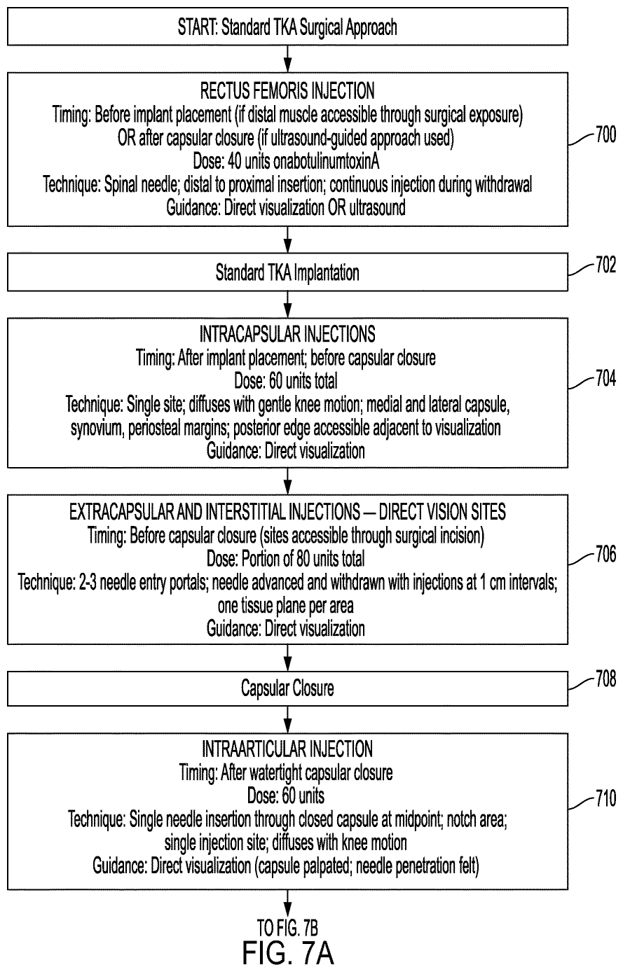

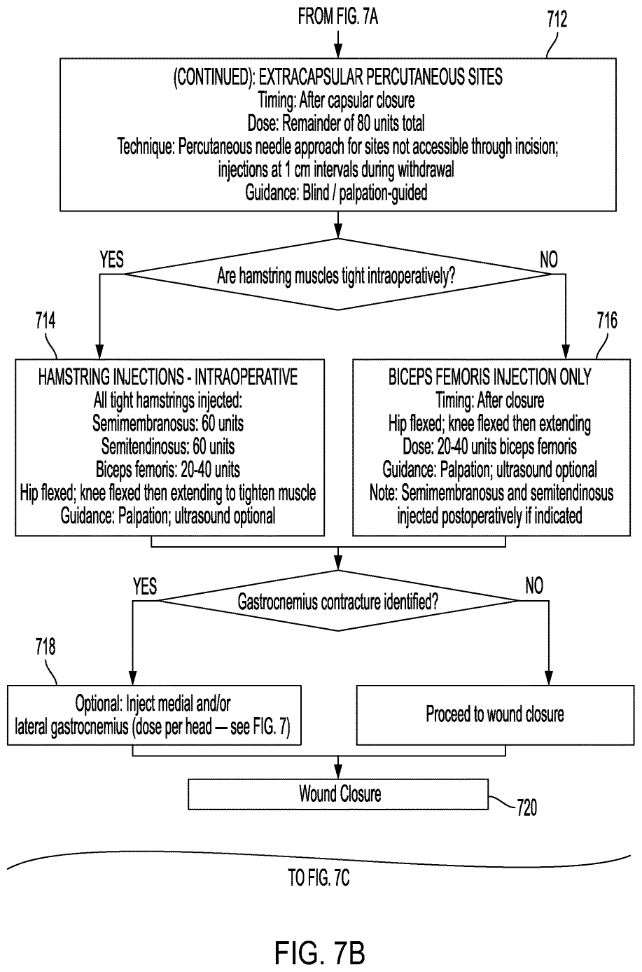

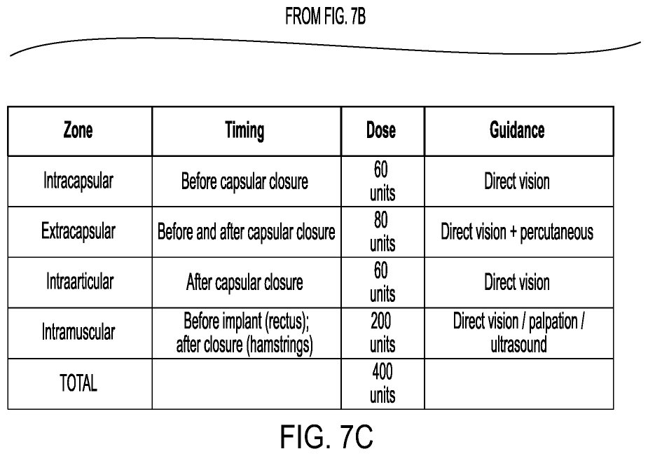

The preferred embodiment of the present invention utilizes a total dose of approximately 400 units of onabotulinumtoxinA (or equivalent) per operative procedure, dissolved in approximately 10 cc of sterile preservative-free injectable normal saline at a concentration of 40 units per cc, distributed across four injection zones as depicted in FIG. 6. Approximately 60 units are administered to the intraarticular zone, approximately 60 units to the intracapsular zone, approximately 80 units to the extracapsular and interstitial zone, and approximately 200 units to the intramuscular zone, for a total of approximately 400 units. The total dose of approximately 400 units has been selected based on the clinical experience of the inventor and the following quantitative rationale: 400 units provides a sufficient number of active toxin molecules (approximately 4 pikamoles) to achieve effective SNARE protein cleavage across the multiple target zones; the 10 cc total volume is appropriate for controlled distribution across the four injection zones without excessive diffusion; the intramuscular zone doses are based on the spasticity literature and the neuromuscular junction density-based dosing principles established in the inventor's prior work (Koman, U.S. Pat. Nos. 8,506,970 and 8,632,786); and the zonal doses reflect the surgeon's ability to deliver the toxin effectively within each zone and the FDA-recommended single-session safety ceiling. In alternative embodiments, the total dose may range from about 200 units to about 500 units, with zone-by-zone distribution maintained in proportional ratios. Equivalent dosing for alternative commercial preparations is provided in FIG. 7; dosing for abobotulinumtoxinA and rimabotulinumtoxinB represents theoretical conversions that have not been clinically verified by the inventor for this specific indication.

The injection protocol is designed to be integrated into the standard TKA surgical workflow with minimal disruption, adding approximately 10 minutes to the standard operative time, as depicted in the flowchart of FIG. 7. At step 700, the distal rectus femoris 152, 153 is accessible through the surgical exposure, the rectus femoris 152 injection is performed before implant placement; otherwise it is deferred until after capsular closure and performed percutaneously under ultrasound guidance. Standard TKA implantation then proceeds at step 702 in the surgeon's routine sequence. With the capsule open following implant placement, intracapsular injections are administered to the synovial tissue 143 under direct vision at step 704, or alternatively during initial approach if clinical circumstances favor earlier administration. Extracapsular and interstitial injections to sites accessible through the surgical exposure are then performed at step 706 using the advance-and-withdraw technique described above. Following standard watertight capsular closure at step 708, the intraarticular injection is administered at step 710 through the closed capsule at the midpoint notch region. Any extracapsular injection sites not accessible through the surgical incision 136 are completed via percutaneous needle approach at step 712. The hamstring injections are then performed at step 714 with the patient's hip flexed and the knee first flexed then gently extended; biceps femoris 158 injection is performed at step 716 in all standard cases, and semimembranosus 173 and semitendinosus 167 injections are performed at this time if either muscle was found to be tight on intraoperative assessment, or deferred to the early postoperative period under ultrasound guidance if not. When gastrocnemius contracture has been identified, gastrocnemius injection of the medial head 176 and/or lateral head 177 is performed as described above at step 718. Standard wound closure then proceeds at step 720 in the surgeon's routine manner.

Practitioners administering botulinum toxin according to the methods disclosed herein should consult and adhere to the complete contraindications, warnings, precautions, and adverse event information set forth in the current FDA-approved labeling for the specific botulinum toxin preparation to be used. Of particular relevance in the perioperative TKA setting, aminoglycoside antibiotics, which are sometimes administered as perioperative prophylaxis, are known to potentiate the neuromuscular blocking effects of botulinum toxin and should be used with caution in patients receiving the methods described herein. Active infection at or near the injection site is a relative contraindication to administration. All injections must be performed in strict accordance with the technique requirements described above, including the avoidance of intravascular injection through awareness of the neurovascular anatomy of the peri-genicular region.

The preferred embodiment described above represents the full four-zone protocol at the 400-unit total dose. In certain alternative embodiments, the methods of the present invention may be modified based on clinical presentation, the nature of the surgical procedure, or the resources available. In patients in whom preoperative and intraoperative assessment reveals balanced muscle forces without significant flexion contracture risk, intramuscular injections may be omitted, with botulinum toxin administered to the intraarticular, intracapsular, and extracapsular/interstitial zones only, for a total dose of approximately 200 units. This three-zone alternative represents a theoretical embodiment that has not been clinically evaluated and outcomes relative to the full four-zone protocol are unknown. In patients at elevated risk of post-TKA stiffness and arthrofibrosis, including those with preoperative flexion contracture, prior arthrotomy, inflammatory arthritis, or significant heterotopic ossification risk, an enhanced protocol may be employed comprising injection of all hamstring muscles 158, 167, 173 intraoperatively regardless of intraoperative muscle tension assessment, together with gastrocnemius injection of the medial head 176 and/or lateral head 177 when contracture is identified, with all doses consistent with those described herein. In patients undergoing revision TKA, greater dissection may be required for adequate visualization. In cases where visualization is insufficient to safely administer posterior injections, the posterior extracapsular zone injections may be omitted, with dosing for accessible zones remaining consistent with the standard protocol. In patients undergoing simultaneous bilateral TKA, the total dose is preferably limited to 200 units per knee (400 units total across both knees) consistent with the FDA-recommended single-session maximum; the bilateral protocol is further limited to the rectus femoris 152 and biceps femoris 158 intramuscular injections only, with posterior injections omitted and unit allocation to each zone decreased proportionally. Given these constraints, staging of the procedure or limitation of botulinum toxin to the more clinically affected knee may be preferable when the full protocol is desired for each limb. In patients undergoing unicondylar knee replacement, the protocol is substantially the same as for primary TKA with the possible omission of posterior injection zones depending on the extent of surgical exposure. In certain embodiments, botulinum toxin may be administered postoperatively as a supplemental treatment in patients who demonstrate early signs of stiffness or arthrofibrosis during rehabilitation. When supplemental injection is indicated in a patient who received the intraoperative protocol, a minimum of six additional weeks following the intraoperative administration should elapse before supplemental injection, reflecting the duration of active neuromuscular blockade, and when performed, postoperative supplemental injections are administered at approximately three months following surgery under ultrasound guidance for hamstring injections and under fluoroscopic or ultrasound guidance for intraarticular injection.

The following examples are provided to illustrate the methods and uses disclosed herein. These examples should not be construed as limiting the scope of the invention.

### Example 1: Prophetic Example-Standard Four-Zone Protocol in Primary TKA for Osteoarthritis

A 67-year-old female patient with end-stage primary knee osteoarthritis presents for elective primary TKA. Preoperative assessment reveals moderate to severe pain with a visual analogue scale (VAS) score in the range of 7 to 9 out of 10, including night pain; limited extension consistent with a clinically significant flexion contracture; flexion limited to approximately 90 degrees or less; and biomechanical alignment within normal parameters. The patient has no contraindications to botulinum toxin administration.

OnabotulinumtoxinA is reconstituted at a concentration of 40 units per cc (four 100-unit vials each reconstituted with 2.5 cc sterile preservative-free saline, yielding 10 cc total). Primary TKA is performed using a standard median parapatellar approach with implantation of a total knee prosthetic system.

Following the standard surgical workflow, the multi-zonal injection protocol is performed in the sequence described above. Prior to implant placement, the distal rectus femoris is identified through the surgical exposure and 40 units are administered using the spinal needle withdrawal technique. Following implant placement and prior to capsular closure, intracapsular injections of 60 units total are administered to the synovial tissue under direct vision. Extracapsular and interstitial injections of 80 units total are distributed circumferentially around the peri-genicular region at approximately 1 to 2 cm intervals using 2 to 3 needle portals, targeting the genicular nerve exit and entry points. Watertight capsular closure is performed. Intraarticular injection of 60 units is administered through the closed capsule under direct palpation at the midpoint notch region. Biceps femoris injection of 40 units is performed percutaneously with the hip flexed and the knee flexed then extending to place the muscle under stretch. Wound closure is then completed in the standard manner.

Postoperatively, the patient is expected to demonstrate a meaningfully reduced VAS pain score consistent with substantial pain relief; minimal stiffness during early rehabilitation; progressive achievement of range of motion rehabilitation goals during the early postoperative period; and an overall clinical course expected to be improved relative to patients with comparable preoperative presentation who did not receive the intraoperative botulinum toxin protocol, based on the inventor's clinical experience with the method.

### Example 2: Prophetic Example-Enhanced Protocol in High-Risk Patient With Preoperative Flexion Contracture

A patient with end-stage knee osteoarthritis and a significant preoperative flexion contracture presents for primary TKA. The preoperative assessment identifies this patient as high-risk for post-TKA stiffness and arthrofibrosis based on the degree of preoperative contracture.

In this case, the enhanced protocol described above is employed, in which all three hamstring muscles are injected intraoperatively at the doses described above, without deferring the semimembranosus and semitendinosus injections to the postoperative period. In most cases, a significant portion of the preoperative flexion contracture is expected to be corrected by the bony resection and alignment achieved during the standard TKA procedure itself; the intraoperative botulinum toxin protocol is designed to address the residual risk of re-developing contracture during the recovery and rehabilitation period by preemptively treating all contributing hamstring muscles at the time of surgery.

Postoperatively, the patient is expected to demonstrate a clinical course consistent with a reduced risk of progressive stiffness and arthrofibrosis during rehabilitation, relative to patients with a comparable degree of preoperative contracture and arthrofibrosis risk who did not receive the intraoperative botulinum toxin protocol, based on the inventor's clinical experience with the enhanced protocol in high-risk patients.

### Example 3: Prophetic Example-Postoperative Supplemental Injection for Early Stiffness

A patient who received the intraoperative four-zone protocol at the time of primary TKA presents approximately nine weeks postoperatively with limited knee flexion that is not improving with physical therapy. Because at least six weeks have elapsed since the intraoperative administration, a meaningful portion of the active neuromuscular blockade from the intraoperative dose may have resolved or diminished sufficiently to permit re-injection, and postoperative supplemental injection is considered.

Ultrasound-guided supplemental injection of the hamstring muscle group is performed at doses consistent with the standard protocol, targeting the muscles clinically assessed as contributing to the residual flexion limitation based on the surgeon's evaluation.

Expected outcomes include progressive improvement in flexion with continued physical therapy following the supplemental injection.

What is claimed is:

1. A method for treating or preventing postoperative pain, inflammation, stiffness, or arthrofibrosis following total knee arthroplasty (TKA), or for balancing muscle forces across a reconstructed knee to improve postoperative range of motion and long-term functional outcomes, comprising administering a botulinum toxin intraoperatively during a TKA surgical procedure to one or more target locations within or surrounding a peri-genicular region of the reconstructed knee, at or prior to the time of surgical wound closure, so as to establish a pharmacological environment that preemptively addresses one or more of postoperative pain, inflammation, stiffness, and arthrofibrosis before postoperative inflammatory and fibrotic cascades become established.

2. The method of claim 1, wherein the botulinum toxin is administered to two or more of: an intraarticular zone, an intracapsular zone, an extracapsular and interstitial zone, and an intramuscular zone.

3. The method of claim 2, wherein the botulinum toxin is administered to all four of the intraarticular zone, the intracapsular zone, the extracapsular and interstitial zone, and the intramuscular zone.

4. The method of claim 2, further comprising administering the botulinum toxin to both the intraarticular zone and the intracapsular zone, in the absence of intramuscular injection, in patients in whom intraoperative assessment reveals balanced muscle forces across the knee without significant flexion contracture risk.

5. The method of claim 1, wherein a total dose of botulinum toxin is from about 200 units to about 500 units of onabotulinumtoxinA or an equivalent.

6. The method of claim 5, wherein the total dose is approximately 400 units dissolved in approximately 10 cc of sterile preservative-free injectable 0.9% sodium chloride solution at a concentration of approximately 40 units per cc.

7. The method of claim 3, wherein a total dose of approximately 400 units is distributed as approximately 60 units to the intraarticular zone, approximately 60 units to the intracapsular zone, approximately 80 units to the extracapsular and interstitial zone, and approximately 200 units to the intramuscular zone.

8. The method of claim 1, wherein an intraarticular injection is administered as a single injection through a closed joint capsule following watertight capsular closure, at the midpoint of the watertight capsular closure in a notch region of the knee joint, the toxin diffusing throughout the intraarticular joint space with subsequent knee range of motion.

9. The method of claim 1, wherein an intracapsular injection is administered to synovial tissue under direct surgical visualization, at a time selected from: during an initial surgical approach before implant placement, at the time of capsular closure, or both.

10. The method of claim 1, wherein extracapsular and interstitial injections are administered to target one or more of: a superior lateral genicular nerve zone, a superior medial genicular nerve zone, an inferior lateral genicular nerve zone, an inferior medial genicular nerve zone, an infrapatellar branch of the saphenous nerve zone, and a common and recurrent fibular nerve branch zone.

11. The method of claim 10, wherein the extracapsular and interstitial injections are directed to terminal nerve endings of a genicular nerve network for the purpose of inhibiting peripheral release of pain-associated neuropeptides including substance P, glutamate, and calcitonin gene-related peptide (CGRP).

12. The method of claim 10, wherein the extracapsular and interstitial injections are performed using an advance-and-withdraw technique comprising inserting a needle at an entry portal, advancing the needle to a distal extent of an injection zone, aspirating to confirm non-intravascular needle position, and injecting botulinum toxin continuously or at approximately 1 to 2 cm intervals during controlled needle withdrawal.

13. The method of claim 12, wherein the advance-and-withdraw technique is performed through 2 to 3 needle entry portals to provide circumferential coverage of an extracapsular peri-genicular zone.

14. The method of claim 1, wherein intramuscular injections are administered to one or more of: the rectus femoris, biceps femoris, semimembranosus, semitendinosus, gracilis, medial gastrocnemius, and lateral gastrocnemius muscles.

15. The method of claim 14, wherein the intramuscular injections comprise administration to the rectus femoris and biceps femoris muscles in all cases, and further comprise administration to the semimembranosus and semitendinosus muscles when those muscles are determined to be tight on intraoperative assessment.

16. The method of claim 14, wherein a rectus femoris injection is performed by inserting a spinal needle at a distal muscle belly under direct surgical visualization or ultrasound guidance, advancing the needle from distal to proximal along longitudinal neuromuscular junction bands of the muscle, and administering botulinum toxin as a continuous slow injection during controlled needle withdrawal.

17. The method of claim 14, wherein a biceps femoris injection is performed with the muscle under stretch, comprising positioning the patient with the hip flexed and the knee flexed then extending, inserting a needle at the most distal accessible portion of the muscle directed from distal to proximal and medial to the common fibular nerve, and administering botulinum toxin at 2 to 3 injection sites.

18. The method of claim 14, further comprising administering botulinum toxin to the medial and/or lateral gastrocnemius when gastrocnemius contracture is identified intraoperatively, comprising approximately 40 units to a medial gastrocnemius head and approximately 20 units to a lateral gastrocnemius head, with those doses reallocated from within an intramuscular zone to maintain a total intramuscular dose of approximately 200 units.

19. The method of claim 14, wherein the dose of botulinum toxin administered to each target muscle in the intramuscular zone is determined based on the number and distribution of neuromuscular junctions within each target muscle, independent of patient body weight.

20. The method of claim 1, wherein all injections are performed at a rate not exceeding approximately 0.5 cc per 3 to 5 seconds, and wherein aspiration is performed at every injection site prior to administration to confirm non-intravascular needle position.

21. The method of claim 1, wherein the botulinum toxin is selected from the group consisting of onabotulinumtoxinA, incobotulinumtoxinA, daxibotulinumtoxinA, abobotulinumtoxinA, rimabotulinumtoxinB, prabotulinumtoxinA, and combinations thereof.

22. The method of claim 1, further comprising inflating a pneumatic tourniquet on an operative extremity during administration of the botulinum toxin to assist in localizing the toxin to the peri-genicular region by limiting venous outflow during injection.

23. The method of claim 1, wherein the method is applied to a simultaneous bilateral TKA procedure, comprising administering a total dose not exceeding approximately 400 units across both knees with approximately 200 units per knee, wherein such administration is limited to an intraarticular zone, an intracapsular zone, an extracapsular and interstitial zone, and rectus femoris and biceps femoris intramuscular injections, with posterior injections omitted.

24. The method of claim 1, wherein the method is applied to a revision TKA procedure, comprising omitting posterior extracapsular zone injections when intraoperative visualization is insufficient to safely access the posterior extracapsular zone injection sites.

25. The method of claim 1, further comprising administering a supplemental postoperative botulinum toxin injection to a hamstring muscle group and/or an intraarticular space when the patient demonstrates inadequate recovery of range of motion during rehabilitation, wherein a minimum of six weeks elapses following intraoperative administration of the botulinum toxin before the supplemental injection is performed.

26. The method of claim 1, wherein the intraoperative administration is prophylactic and configured to preempt initiation of postoperative inflammatory and fibrotic cascades before they become established.

27. A method for performing total knee arthroplasty comprising:
reconstituting approximately 400 units of onabotulinumtoxinA with sterile preservative-free injectable normal saline to yield a concentration of approximately 40 units per cc and a total volume of approximately 10 cc; and
intraoperatively administering the reconstituted botulinum toxin in a four-zone protocol comprising approximately 60 units to the intraarticular zone after watertight capsular closure, approximately 60 units to the intracapsular zone before or during capsular closure, approximately 80 units to the extracapsular and interstitial zone targeting genicular nerve exit and entry points via an advance-and-withdraw needle technique, and approximately 200 units to the intramuscular zone comprising at least the rectus femoris and biceps femoris muscles;
wherein the botulinum toxin administered to the extracapsular and interstitial zone cleaves SNARE proteins at terminal sensory nerve endings to inhibit peripheral release of pain-associated neuropeptides, and the botulinum toxin administered to the intramuscular zone addresses muscle force imbalance contributing to postoperative stiffness and flexion contracture.

28. A kit for the intraoperative administration of botulinum toxin during total knee arthroplasty comprising:
a botulinum toxin preparation in a unit dose of from about 300 units to about 500 units;
sterile preservative-free injectable 0.9% sodium chloride solution for reconstitution to a concentration of approximately 40 units per cc;
one or more sterile syringes;
one or more sterile hypodermic needles suitable for periarticular and intracapsular injection;
a sterile spinal needle suitable for intramuscular injection using a distal-to-proximal advance-and-withdraw technique; and
instructions for intraoperative administration of the botulinum toxin according to a multi-zonal protocol comprising administration to at least an intraarticular zone, an intracapsular zone, an extracapsular and interstitial zone, and an intramuscular zone of a peri-genicular region during a TKA surgical procedure.
